(19) United States
(12) Patent Application Publication
(10) Pub. No.: US 2022/0249174 A1
(43) Pub. Date: Aug. 11, 2022

(54) SURGICAL NAVIGATION SYSTEM, INFORMATION PROCESSING DEVICE AND INFORMATION PROCESSING METHOD

(71) Applicant: FUJIFILM Healthcare Corporation, Kashiwa-shi (JP)

(72) Inventors: Rena SHINOHARA, Chiba (JP); Nobutaka ABE, Chiba (JP)

(21) Appl. No.: 17/578,543

(22) Filed: Jan. 19, 2022

(30) Foreign Application Priority Data
Feb. 10, 2021 (JP) 2021-020036

(51) Int. Cl. A61B 34/20; A61B 34/10; A61B 90/00

(52) U.S. Cl. CPC A61B 34/20; A61B 34/10; A61B 90/37; A61B 2034/744; A61B 2034/2057; A61B 2034/101; A61B 90/39

(57) ABSTRACT

To quickly and accurately register a surgical field image and a preoperative image with each other and display the surgical field image and the preoperative image. The invention extracts sulcus patterns included in the preoperative image, and extracts sulcus patterns included in the surgical field image of a brain of a patient during a surgical operation. The invention extracts, from the sulcus patterns of the preoperative image, a range that matches the sulcus patterns of the surgical field image, and calculates a conversion vector for converting the preoperative image to match the range with the surgical field image. The invention displaces the preoperative image by the conversion vector and displays the preoperative image on a connected display device.

### BACKGROUND OF THE INVENTION

### 1. Field of the Invention

The present invention relates to a surgical navigation system that registers and displays a surgical field image of a microscope and a medical image obtained from a medical image acquisition device.

### 2. Description of the Related Art

Surgical navigation systems have been known for assisting surgeons in performing a surgical operation safely and securely by integrating treatment plan data created before the surgical operation and data acquired during the surgical operation to guide positions and postures of surgical instruments or the like. For example, the surgical navigation system is configured to superimpose and display position information in real space of various medical devices such as surgical instruments detected by a sensor such as a position measuring device on a medical image of a patient captured before a surgical operation by a medical image capturing device such as MRI to assist the surgical operation. As a result, the surgeon can understand a positional relation image between actual positions of the surgical instruments and the medical image, for example, a tumor on the medical image.

In order for the position measuring device or the like to detect the positions of the surgical instruments or the patient in the real space, a marker is attached to the surgical instruments or the patient. When capturing a preoperative medical image, a marker is also attached to the same position of the patient and the image is captured. By associating the position of the marker on the medical image with the position of the marker of the patient, image space coordinates and real space coordinates are associated (registered).

WO2018/012080 (Patent Literature 1) discloses a surgical navigation technique for comparing a predetermined pattern of blood vessels or the like on a preoperative image with a predetermined pattern of blood vessels or the like on an image of a surgical field imaged with a microscope during the surgical operation, and deforming the preoperative image according to the surgical field image to display the preoperative image together with a treatment tool. Specifically, with the surgical navigation device of Patent Literature 1, the target living tissue is a brain, a 3D model (three-dimensional image) of the brain is generated based on an image captured before the surgical operation, and pattern matching is performed between a blood vessel pattern on the surface of the 3D model and a blood vessel pattern included in an image captured during the surgical operation. Based on the pattern matching result, the amount of brain deformation (brain shift) due to craniotomy is calculated by estimating displacements of three-dimensional meshes with a finite element method. The 3D model is deformed based on the calculated deformation amount, and a navigation image with an indication showing a position of the treatment tool is displayed.

In the technique described in Patent Literature 1, the displacement amount of the brain is calculated by performing the pattern matching by using the blood vessel pattern of the image captured before the surgical operation and a blood vessel pattern of a microscopic image of the surgical field after the craniotomy. However, when the preoperative image is captured by Magnetic Resonance Imaging (MRI), the accuracy is low because the blood vessels on the brain surface can not be clearly visualized. Further, since the brain surface is incised after the start of the surgical operation, it is difficult to use the blood vessel pattern to calculate the displacement of the brain during the surgical operation.

Further, the method of placing markers on the surface of the brain during the surgical operation to detect the displacement of the brain is burdensome to the patient and the surgeon.

On the other hand, in an actual surgical operation, with the progress of the surgical operation, living tissue of the patient is cut open and a tumor or the like is excised, and thus the excised tissue is removed, or surrounding tissue is shifted to fill a space where the excised tissue was. Accordingly, an anatomical structure of the patient itself is changed, so it is desirable to sequentially update the images obtained before the surgical operation to reflect the deformation of the brain that occurred during the surgical operation. However, with the technique described in Patent Literature 1, it is difficult to estimate the change in the anatomical structure during the surgical operation and update the preoperative image.

### SUMMARY OF THE INVENTION

An object of the invention is to provide a technique for quickly and accurately registers an image of a surgical field captured in real time with an image captured before a surgical operation without using a special instrument.

To achieve the object described above, a surgical navigation system of the invention includes a preoperative sulcus pattern extraction unit configured to receive a preoperative image captured of the brain of a patient before a surgical operation and extract a sulcus pattern included in the preoperative image, a surgical field sulcus pattern extraction unit configured to receive a surgical field image from a surgical field image capturing device that captures the surgical field image of the brain of the patient during the surgical operation, and extract the sulcus pattern included in the surgical field image, a search unit configured to search for a range of sulcus patterns that matches the sulcus pattern in the surgical field image from the sulcus patterns in the preoperative image, a conversion vector calculation unit configured to calculate a conversion vector that matches the sulcus pattern in the searched range with the sulcus pattern in the surgical field image, and a calculation unit configured to convert coordinates of the preoperative image by using the conversion vector and display the preoperative image on a connected display device.

According to the invention, since the surgical field image and the preoperative image can be quickly and accurately registered with each other and displayed, the progress of the surgical operation can be smoothed and the accuracy of the surgical operation can be improved.

### BRIEF DESCRIPTION OF THE DRAWINGS

FIG. 1 is a diagram showing a hardware configuration of a surgical navigation system according to a first embodiment of the invention.

FIG. 2 is a perspective view of a surgical field image acquisition device (microscope device), a surgical instrument position detection device, and a bed.

FIG. 3 is a functional block diagram of an information acquisition and processing unit of the surgical navigation system according to the first embodiment.

FIG. 4 is a flowchart showing processing operations of the information acquisition and processing unit according to the first embodiment.

FIGS. 5A to 5C are explanatory views showing the processing operations of the information acquisition and processing unit of the surgical navigation system according to the first embodiment.

FIG. 6 is a functional block diagram of the information acquisition and processing unit of the surgical navigation system according to a second embodiment.

FIG. 7 is a flowchart showing processing operations of the information acquisition and processing unit according to the second embodiment.

FIGS. 8A and 8B are illustrative views showing the processing operations of the information acquisition and processing unit according to the second embodiment.

FIG. 9 is a functional block diagram of the information acquisition and processing unit according to a third embodiment.

FIG. 10 is a flowchart showing processing operations of the information acquisition and processing unit according to the third embodiment.

FIG. 11 is an illustrative view showing the processing operations of the information acquisition and processing unit according to the third embodiment.

FIG. 12 is a functional block diagram of the information acquisition and processing unit according to a fourth embodiment.

FIG. 13 is a flowchart showing the processing operations of the information acquisition and processing unit according to the fourth embodiment.

FIG. 14 is an illustrative view showing the processing operations of the information acquisition and processing unit according to the fourth embodiment.

### DESCRIPTION OF THE PREFERRED EMBODIMENTS

Hereinafter, an embodiment of the invention will be described with reference to the drawings. In the following description and the accompanying drawings, components having the same functional configuration are denoted by the same reference numerals, and repeated description thereof will be omitted.

### 1. First Embodiment

A surgical navigation system according to a first embodiment receives a captured preoperative image of a brain of a patient before a surgical operation to extract sulcus patterns included in the preoperative image, while receiving a surgical field image from a surgical field image capturing device that captures the surgical field image of the brain of the patient during the surgical operation to extract the sulcus pattern included in the surgical field image. The surgical navigation system according to the first embodiment then extracts, from a plurality of ranges in the preoperative image, a range including sulcus patterns that match the sulcus patterns of the surgical field image, and calculates a conversion vector for converting the coordinates of the preoperative image to match the range with the surgical field image. Finally, the surgical navigation system according to the first embodiment displaces the preoperative image by the conversion vector and displays the preoperative image on a connected display device.

### 1-1. Configuration

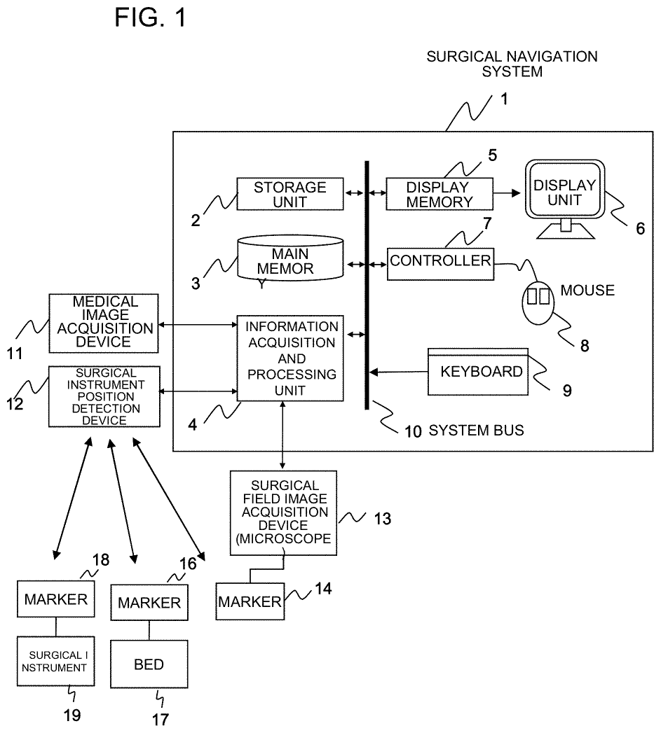

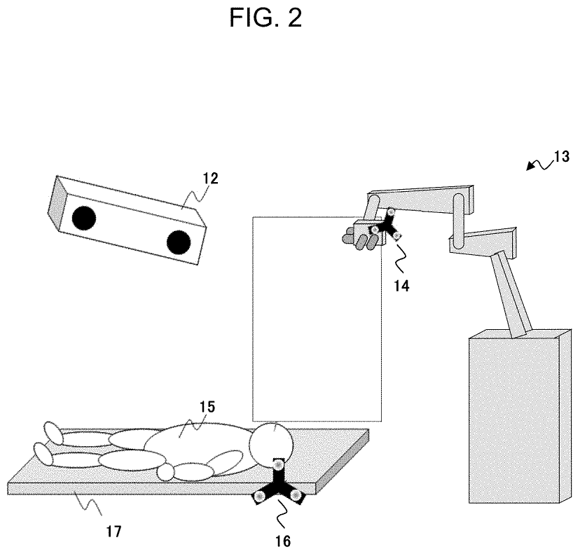

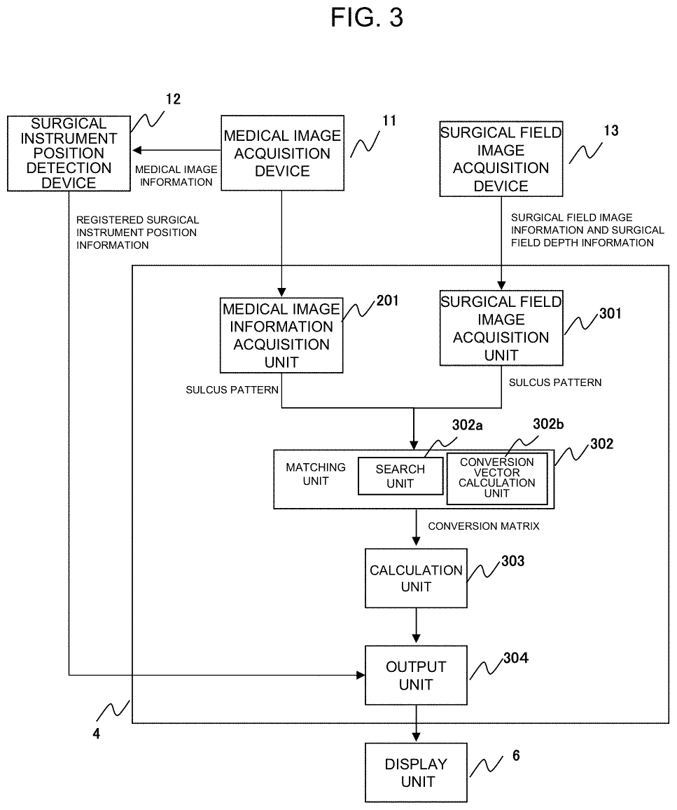

FIG. 1 is a block diagram showing a hardware configuration of a surgical navigation system 1 according to the embodiment. FIG. 2 is a perspective view showing a surgical instrument position detection device 12, a surgical field image acquisition device 13, and a bed 17. FIG. 3 is a functional block diagram of an information acquisition and processing unit 4 of the surgical navigation system 1.

As shown in FIG. 1, the surgical navigation system 1 according to the first embodiment is connected to a medical image acquisition device 11, the surgical instrument position detection device 12, and the surgical field image acquisition device 13, registers and displays a preoperative medical image (preoperative image) of a patient received from the medical image acquisition device 11 and a surgical field image during a surgical operation (surgical field image) captured by the surgical field image acquisition device 13. In that case, a mark showing the position of a surgical instrument is displayed on the medical image.

The surgical navigation system 1 includes the information acquisition and processing unit 4, a storage unit 2, a main memory 3, a display memory 5 to which a display unit 6 is connected, the display unit 6, a controller 7 to which a mouse 8 is connected, and a keyboard 9, which are connected by a system bus 10 so as to be able to transmit and receive signals. Here, “be able to transmit and receive signals” indicates a state of being capable of transmitting and receiving a signal to and from each other or from one to the other regardless of whether a connection is electrically or optically wired or wireless.

The medical image acquisition device 11, the surgical instrument position detection device 12, and the surgical field image acquisition device 13 are connected to the information acquisition and processing unit 4 so as to be able to transmit and receive signals.

The medical image acquisition device 11 is an image capturing device such as MRI, CT, and an ultrasonic image capturing device, and captures a three-dimensional image of the patient as the medical image.

The surgical instrument position detection device 12 is a device that detects the real space positions of a surgical instrument 19, a patient 15 lying on the bed 17, and the surgical field image acquisition device 13, and it may be an optical detection device (stereo camera) or a magnetic detection device (magnetic sensor). Here, a stereo camera is used as the surgical instrument position detection device 12.

The surgical instrument 19 is an instrument for performing incising or excising on a patient, for example, an electric scalpel such as a monopolar or a bipolar. A marker 18 is fixed to the surgical instrument 19, and a position in the real space is detected by the surgical instrument position detection device 12.

The surgical field image acquisition device 13 is a device that captures and acquires an image of the surgical field of the patient, in which a surgical microscope is used. It is premised that the surgical field image acquisition device 13 has two cameras on the left and right as an optical system capable of performing stereo viewing. As shown in FIG. 2, a surgical field image position information acquisition unit (for example, a marker) 14 is attached to the surgical field image acquisition device (surgical microscope) 13, and a position thereof in the real space is detected by the surgical instrument position detection device 12.

A patient position information acquisition unit (marker) 16 is also attached to the bed 17 on which the patient 15 is lying, and a position thereof is detected by the surgical instrument position detection device 12. Accordingly, it is possible to detect the position of the patient lying on the bed 17 at a predetermined position.

The information acquisition and processing unit 4, as shown in the functional block diagram in FIG. 3, includes a surgical field image acquisition unit 301 that acquires the surgical field image from the surgical field image acquisition device 13, and extracts the sulcus patterns, a matching unit 302 that compares the sulcus patterns of the medical image and the sulcus patterns of the surgical field image to obtain the conversion vector, a calculation unit 303 that converts the coordinates of the preoperative image with the obtained conversion vector, and an output unit 304. The matching unit 302 includes a search unit 302a and a conversion vector calculation unit 302b. The search unit 302a searches for a range of the sulcus patterns that matches the sulcus patterns of the surgical field image from the sulcus patterns in the preoperative image. The conversion vector calculation unit 302b calculates a conversion vector that matches the sulcus patterns in the searched range with the sulcus patterns of the surgical field image.

The information acquisition and processing unit 4 includes a CPU (not shown), and the CPU achieves functions of the blocks (301 to 304) with software by loading a program pre-stored in the storage unit 2 and data necessary for executing the program into the main memory 3 and executing the program. The information acquisition and processing unit 4 can also achieve a part or all of the functions of the blocks (301 to 304) by hardware. For example, a circuit design may be performed using a custom IC such as an application specific integrated circuit (ASIC) or a programmable IC such as a field-programmable gate array (FPGA) so as to achieve the functions of the blocks (301 to 304).

The storage unit 2 is a hard disk or the like. Further, the storage unit 2 may be a device that exchanges data with a portable recording medium such as a flexible disk, an optical (magnetic) disk, a ZIP memory, or a USB memory.

The main memory 3 stores a progress of the program and arithmetic processing executed by the information acquisition and processing unit 4.

The display memory 5 temporarily stores display data to be displayed on the display unit 6 such as a liquid crystal display or a Cathode Ray Tube (CRT) display.

The mouse 8 and the keyboard 9 are operation devices with which an operator gives an operation instruction to the system 1. The mouse 8 may be another pointing device such as a trackpad or a trackball.

The controller 7 detects a state of the mouse 8, acquires a position of a mouse pointer on the display unit 6, and outputs the acquired position information and the like to the information acquisition and processing unit 4.

### 1-2. Processing

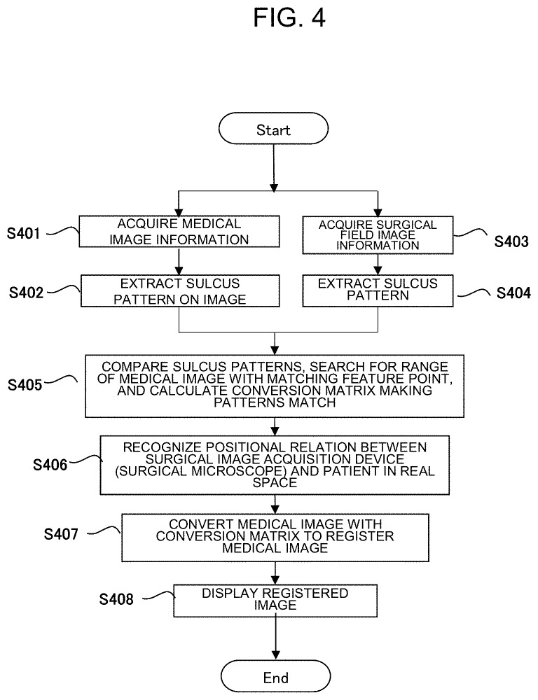

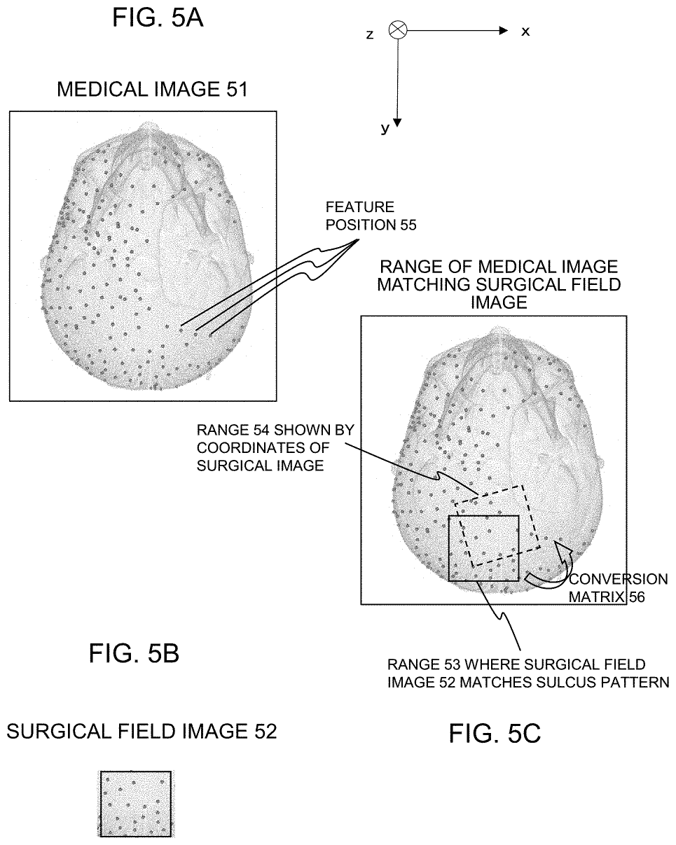

Hereinafter, processing operations of each unit of the information acquisition and processing unit 4 will be specifically described with reference to a flow of FIG. 4 and an image example of FIGS. 5A-5C.

### (Step S401)

A medical image information acquisition unit 201 of the information acquisition and processing unit 4 acquires a medical image 51 from the medical image acquisition device 11 via a Local Area Network (LAN) or the like (see FIG. 5A). Specifically, the medical image information acquisition unit 201 acquires a three-dimensional medical image such as an MRI image or an X-ray CT image from the medical image acquisition device 11, generates a surface-rendering (SR) image in a plurality of directions by image processing, and sets the surface-rendering image as the medical image 51.

### (Step S402)

The medical image information acquisition unit 201 acquires a position of a groove as a feature position 55 on the medical image 51 (see FIG. 5A). For example, the medical image information acquisition unit 201 performs smoothing processing on the medical image 51 and acquires average depth information for each pixel. When a difference is larger than a preset threshold as compared with the depth information before the smoothing, it is deemed that there is a groove, and the medical image information acquisition unit 201 extracts the groove part as a feature position 55 (feature point, that is, a sulcus). Hereinafter, a plurality of feature positions 55 (positions of grooves) are also referred to as sulcus patterns.

### (Step S403)

The surgical field image acquisition unit 301 acquires a current surgical field image (still image) 52 from the left and right cameras of the surgical field image acquisition device 13.

### (Step S404)

When a distance between the left and right cameras of the surgical field image acquisition device 13 is B, a focal length is F, a distance to an object to be captured is Z, and a parallax in the images of the left and right cameras is D, the surgical field image acquisition unit 301 calculates the value of Z by Z=B×F/D for each pixel. The surgical field image acquisition unit 301 acquires three-dimensional position information of each pixel of the surgical field image 52 from the pixel position and the distance to the object to be captured.

By performing smoothing processing on depth information (z direction) of the three-dimensional position information of the surgical field image 52, the surgical field image acquisition unit 301 acquires average depth information for each pixel, and when a difference is larger than a preset threshold as compared with the depth information before the smoothing, it is deemed that there is a groove, and the surgical field image acquisition unit 301 extracts the groove part as the feature position (feature point, that is, sulcus pattern) (FIG. 5B).

### (Step S405)

The search unit 302a of the matching unit 302 compares the sulcus pattern (feature point) of the medical image 51 extracted in step S402 with the sulcus pattern (feature point) of the surgical field image 52 extracted in step S404, and searches for a range 53 of the medical image 51 that best matches the sulcus pattern of the surgical field image 52. The conversion vector calculation unit 302b uses an iterative closest point (ICP) algorithm to perform iterative calculations for matching a point cloud of the sulcus pattern (FIG. 5B) in the range 53 of the medical image 51 and a point cloud of the sulcus pattern (feature point) of the surgical field image 52, obtains a translation vector and a rotation matrix, and uses the translation vector and the rotation matrix as a conversion matrix.

### (Step S406)

The calculation unit 303 receives a position of the surgical field image position information acquisition unit (marker) 14 attached to the surgical field image acquisition device (surgical microscope) 13 and a position of the patient position information acquisition unit (marker attached to the bed) 16 from the surgical instrument position detection device 12. As a result, the calculation unit 303 recognizes positions of the surgical field image acquisition device (surgical microscope) 13 and the patient 15 in the real space, respectively.

### (Step S407)

The calculation unit 303 converts the medical image by using the conversion matrix obtained in step S405 as shown in FIG. 5C. As a result, the registration of the medical image space coordinates and the real space coordinates is performed.

### (Step S408)

The calculation unit 303 receives the position of the marker 18 of the surgical instrument 19 acquired by the surgical instrument position detection device 12, and recognizes the position of the surgical instrument 19.

The output unit 304 displays the medical image after the registration in step S407. In that case, a mark such as an arrow or a circle indicating the position of the surgical instrument 19 is displayed on the medical image.

### 1-3. Effects

According to the first embodiment, the following effects can be obtained.

It is possible to quickly achieve medical image registration by using the sulci of the medical image and the surgical field image without the need for special instruments and operations, and therefore the stress and burden on surgeon can be reduced.

### 2. Second Embodiment

A surgical navigation system of a second embodiment will be described.

In the second embodiment, by using a medical image in which medical image space coordinates and real space coordinates have already been superimposed and a surgical field image, the sulcus patterns of the surgical field image and the medical image are compared in real time during the surgical operation, and the medical image is updated (deformed) according to an anatomical structure of the patient acquired from the surgical field image.

That is, the surgical navigation system according to the second embodiment receives a captured preoperative image of a brain of a patient before a surgical operation to extract sulcus patterns included in the preoperative image, while receiving a surgical field image from a surgical field image capturing device that captures the surgical field image of the brain of the patient during the surgical operation to extract the sulcus patterns included in the surgical field image. The surgical navigation system according to the second embodiment then extracts, from a plurality of ranges in the preoperative image, a range including sulcus patterns that best match the sulcus patterns of the surgical field image, and deforms the preoperative image (in the depth direction) to match the range with the surgical field image. The surgical navigation system according to the second embodiment displays the deformed preoperative image on the connected display device.

### 2-1. Configuration

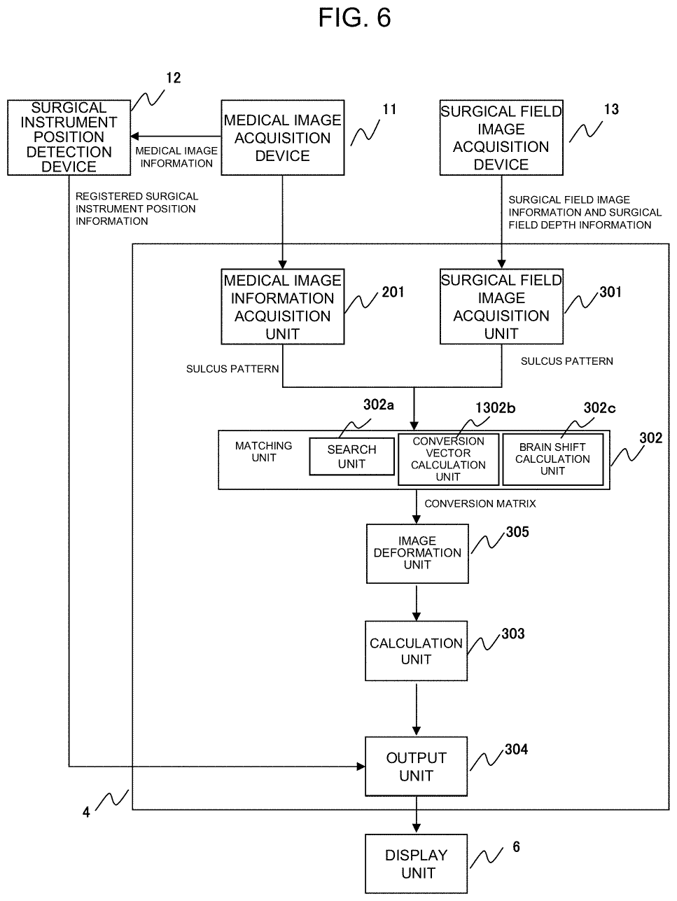

As shown in FIG. 6, the configuration of the second embodiment is different from that of the first embodiment in that the information acquisition and processing unit 4 includes an image deformation unit 305 that deforms an image. Further, the matching unit 302 is different from that of the first embodiment in that a displacement vector calculation unit 1302 is provided instead of the conversion vector calculation unit 302b, and a brain shift calculation unit 302c is further provided. Since the other configurations are the same as those in the first embodiment, a description thereof will be omitted.

### 2-2. Processing

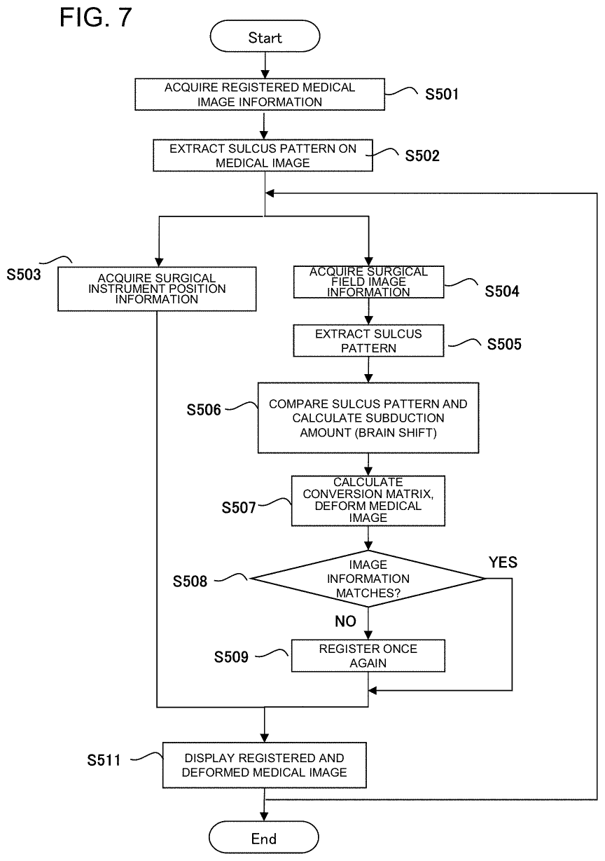

Hereinafter, processing operation of each unit of the information acquisition and processing unit 4 will be specifically described with reference to a flow of FIG. 7.

### (Step S501)

The medical image information acquisition unit 201 of the information acquisition and processing unit 4 acquires the medical image 51 in which the medical image space coordinates and the real space coordinates have already been registered with each other from the medical image acquisition device 11.

### (Step S502)

The medical image information acquisition unit 201 acquires the sulcus patterns on the medical image 51 in the same manner as in step S402 of the first embodiment.

### (Step S503)

The surgical instrument position detection device 12 detects a surgical instrument position in the real space coordinates.

### (Step S504)

The surgical field image acquisition unit 301 sequentially acquires the surgical field images from the surgical field image acquisition device (surgical microscope) 13 before and during the surgical operation.

### (Step S505)

The surgical field image acquisition unit 301 extracts the sulcus patterns of the preoperative surgical field image, as in step S404 of the first embodiment.

### (Step S506)

The search unit 302a of the matching unit 302, as in step S405 of the first embodiment, compares the sulcus patterns of the medical image 51 extracted in step S502 with the sulcus patterns (feature points) of the surgical field image 52 extracted in step S505, and search for a range 53 of the medical image 51 that best matches the sulcus patterns of the surgical field image 52.

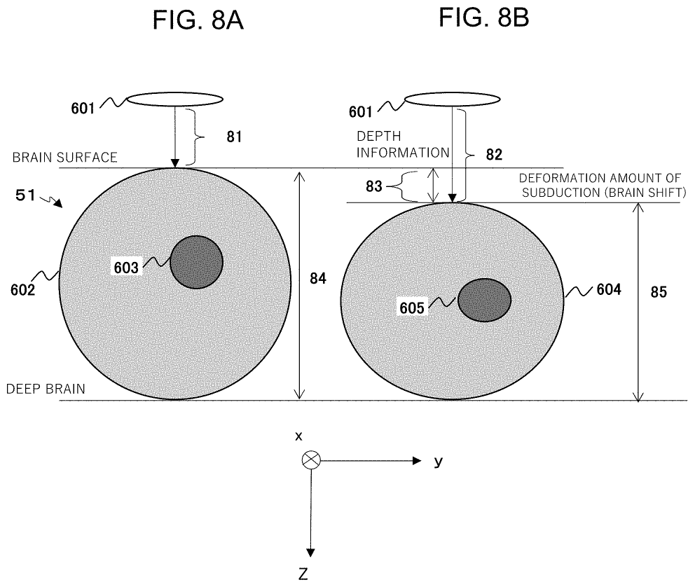

Next, the brain shift calculation unit 302c obtains depth information 81 of the preoperative medical image 51 (FIG. 8A), then obtains depth information 82 in FIG. 8B from the intraoperative surgical field image 52, and calculates a difference (subduction amount: brain shift) 83 between the two images.

The depth information 81 and 82 in FIGS. 8A and 8B are the distances between a camera 601 of the surgical field image acquisition device (microscope) 13 and a surface of a tissue (brain) 602 of the patient 15.

Specifically, the brain shift calculation unit 302c obtains the depth information 81 and 82 of the medical images 51 and 52 by calculating the distance Z from the camera to the object to be captured (brain surface) in the same manner as in step S404 of the first embodiment.

The difference 83 between the depth information 81 and 82 calculated in step S506 shows the deformation amount (brain shift) of the tissue 602 including a lesion 603 before the surgical operation and a tissue 604 with a lesion 605 after partial removal of the lesion 603.

### (Step S507)

The calculation unit 303 deforms the medical image 51 in the depth direction and within the plane, and obtains a displacement field matrix that matches the medical image 51 with the surgical field image 52 by using an affine transformation. Specifically, first, the calculation unit 303 obtains depth information 85 of the tissue 604 from a difference between depth information 84 of the tissue 602 calculated from the preoperative medical image 51 and the deformation amount 83 of the subduction. Then, starting from the deep brain, the calculation unit 303 obtains the displacement field matrix for deforming the medical image 51 by applying the affine transformation by using a ratio of the depth information 84 of the tissue 602 to the depth information 85 of the tissue 604.

Accordingly, this makes it possible to obtain a conversion matrix to match a brain shape that was a shape as shown in FIG. 8A with a brain shape after subduction (brain shift) in the depth direction as shown in FIG. 8B by craniotomy or lesion removal.

### (Step S508)

The image deformation unit 305 transforms the medical image 51 by using the conversion matrix obtained in step S507. This produces a medical image 51 that matches the real-time anatomical structure of the patient.

### (Step S509)

The image deformation unit 305 determines whether the deformed medical image 51 produced in step S508 and the medical image 51 acquired in step 501 match. For example, the feature points (sulcus patterns) of the images are binarized and compared to determine whether the images match.

### (Step S510)

When the image deformation unit 305 determines in step S509 that the image information does not match, the image deformation unit 305 updates the image information and registers the space coordinates and the real space coordinates of the medical image 51 with each other once again.

### (Step S511)

The output unit 304 displays the registered and deformed medical image 51. At this time, a mark such as an arrow or a circle indicating a position of the surgical instrument 19 acquired in step S508 is displayed in the deformed medical image 51.

### 2-3. Effects

According to the second embodiment, the following effects can be obtained. That is, with the procedure of the surgical incision or excision, the medical image captured before the surgical operation becomes different from the anatomical structure of the living tissue of the current patient, and the image deformation unit 305 is capable of transforming the medical image to correct the difference in real time. Since the surgeon can confirm the position of the tumor by looking at the medical image after the deformation, it is possible to realize a highly accurate surgical operation.

### 3. Third Embodiment

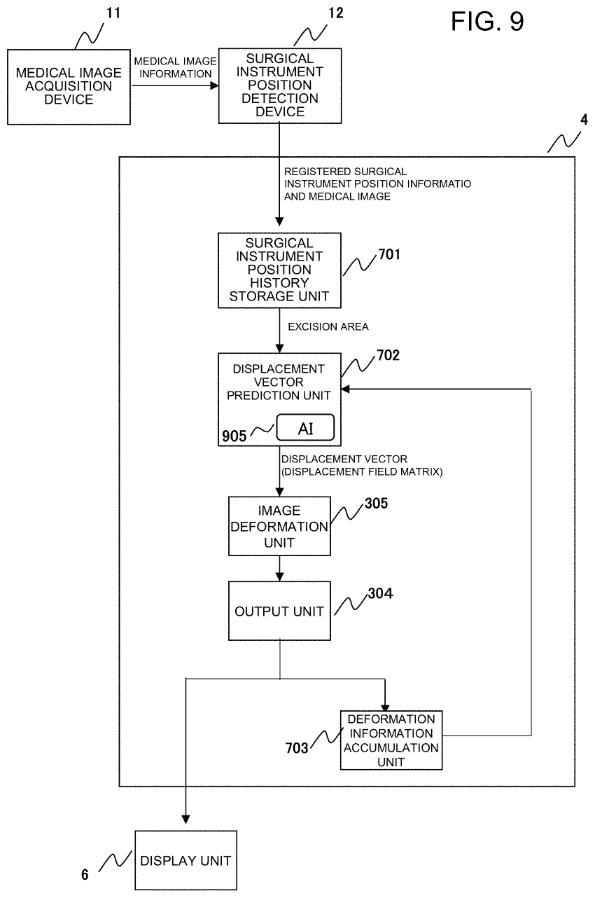

A surgical navigation system of a third embodiment will be described with reference to FIGS. 9 to 11.

In the third embodiment, a displacement vector is predicted and an image is transformed to fit an actual anatomical structure of a patient. Further, a displacement vector prediction function is updated after comparing accumulated displacement vector prediction information with microscope image information.

That is, the surgical navigation system receives a captured preoperative image of a brain of a patient before a surgical operation, and also receives position data of a surgical instrument in chronological order. The surgical navigation system calculates a range of living tissue removed by the surgical instrument as an excision area from chronological position data of the surgical instrument. The surgical navigation system uses the excision area to predict a displacement vector indicating the deformation that occurs in the preoperative image by calculation, and deforms the preoperative image by the obtained displacement vector. The surgical navigation system displays the deformed preoperative image on the connected display device.

### 3-1. Configuration

A configuration of the third embodiment is different from that of the first embodiment in a configuration of the information acquisition and processing unit 4.

The information acquisition and processing unit 4 includes, as shown in a functional block diagram of FIG. 9, a surgical instrument position history storage unit 701 that acquires surgical instrument position information that has been registered with a medical image and the medical image from the surgical instrument position detection device 12, a displacement vector prediction unit 702 that predicts brain shift based on a trajectory of the surgical instrument, the image deformation unit 305 that deforms the image based on the prediction, the output unit 304 that outputs the image, and a deformation information accumulation unit 703 that accumulates deformation information.

The displacement vector prediction unit 702 uses the preoperative image (for example, an MRI image) and the removed (excised) area as input data, a displacement field matrix as teacher data and is equipped with a learned learning model (artificial intelligence algorithm) 905. Accordingly, the displacement vector prediction unit 702 is capable of predicting the deformation due to the brain shift by inputting an actual preoperative image (medical image 51) and an excision area calculated from a surgical instrument position into the learning model, and outputting the displacement field matrix.

As the artificial intelligence algorithm, it is preferable to use an AI algorithm for deep learning, such as a convolutional neural network. Specifically, well-known AI algorithms such as U-net, Seg-net, or DenseNet can be used as the AI algorithm.

In the learning process, the input data is input to an artificial intelligence algorithm before learning, and output prediction data is compared with the teacher data. By feeding back the comparison result to the artificial intelligence algorithm to repeat a modification of the algorithm, the artificial intelligence algorithm is optimized so that an error between the prediction data and the teacher data is minimized.

### 3-2. Processing

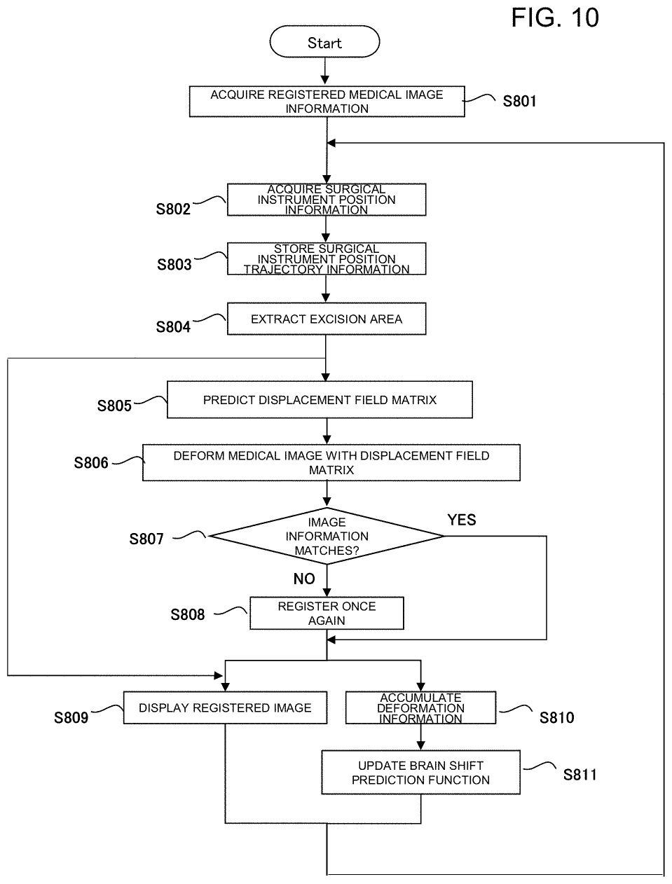

Hereinafter, the processing operation of each part of the information acquisition and processing unit 4 will be specifically described with reference to the flow of FIG. 10.

### (Step S801)

The surgical instrument position detection device 12 acquires the medical image 51 in which the medical image space coordinates and the real space coordinates have already been registered with each other from the medical image acquisition device 11 via an LAN or the like.

### (Step S802)

The surgical instrument position history storage unit 701 acquires the surgical instrument position information from the surgical instrument position detection device 12.

### (Step S803)

The surgical instrument position history storage unit 701 saves the surgical instrument position information acquired in step S802 as a trajectory.

### (Step S804)

The surgical instrument position history storage unit 701 calculates the excision area based on the trajectory information acquired in step S803. For example, the trajectory through which the surgical instrument (electric scalpel or the like) has passed or an area 902 surrounded by the trajectory is determined to be an excised area.

### (Step S805)

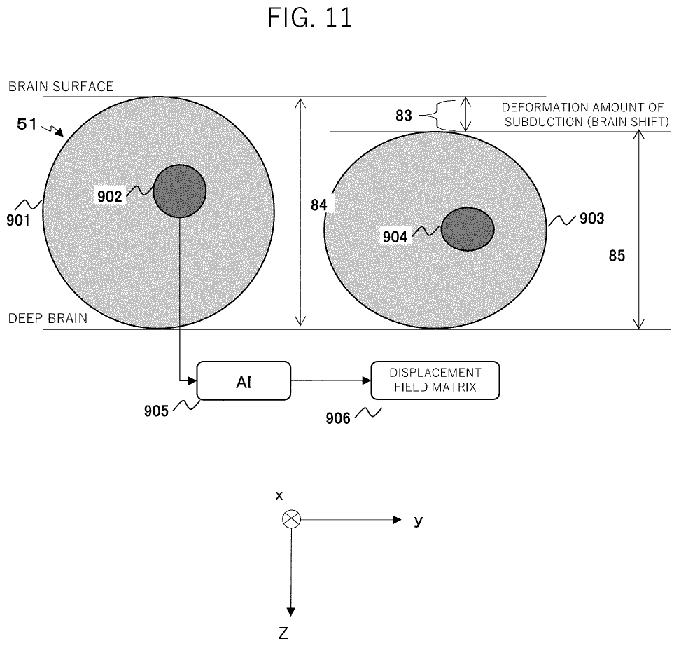

The displacement vector prediction unit 702 inputs the excision area 902 calculated in step S804 and the medical image 51 acquired in step S801 into the learned learning model 905 to obtain a displacement field matrix 906 to be output by the learned learning model 905 (see FIG. 11).

### (Step S806)

The image deformation unit 304 deforms the medical image 51 by applying the displacement field matrix (deformation vector) 906 obtained in step S805 to the medical image 51 acquired in step S801. Specifically, the image deformation unit 304 multiplies the data of the medical image 51 arranged in a matrix format by the displacement field matrix 906 to produce the medical image after the brain shift.

Accordingly, as shown in FIG. 11, a part of the lesion 902 of the tissue 901 of the medical image 51 is removed, a tissue 903 and a lesion 904 after the lesion removal are deformed from the tissue 903 and the lesion 904 before the removal, and the medical image after the brain shift in which the brain surface is subducted can be obtained.

### (Step S807)

The image deformation unit 304 of the information acquisition and processing unit 4 determines whether the deformed medical image generated in step S806 and the medical image 51 acquired in step 801 match. For example, the feature points (sulcus patterns) of the images are binarized and compared to determine whether the images match.

### (Step S808)

When it is determined that the deformed medical image generated in step S806 and the medical image 51 acquired in step S801 do not match, the image deformation unit 304 registers the space coordinates and the real space coordinates of the medical image 51 with each other once again.

### (Step S809)

The output unit 304 registers and outputs a mark showing the position of the surgical instrument on the superimposed medical image, and then displays the mark on the display unit 6.

### (Step S810)

Further, the deformation information accumulation unit 703 accumulates image deformation information acquired in step S806.

### (Step S811)

The displacement vector prediction unit 702 uses the image deformation information (simulation result) accumulated in step S810 to update the displacement vector prediction function for performing the displacement vector prediction with higher accuracy. Specifically, a displacement field matrix is obtained to match the image captured by the medical image capturing device such as an MRI after the surgical operation with the medical image 51 accumulated in step S810, and the learning model 905 is relearned using this displacement field matrix as the output data (teacher data). The input data are the medical image 51 and the excision area obtained in step S804.

### 3-3. Effects

According to the third embodiment, the following effects can be obtained.

With the procedure of the surgical incision or excision, the preoperative medical image and the anatomical structure of the patient during the surgical operation become different, but it is possible to predict the difference, transform the medical image by calculation, and display the deformed medical image together with the position of the surgical instrument. Therefore, the surgeon can confirm the position of the tumor by looking at the medical image after the deformation, and thus it is possible to realize a highly accurate surgical operation.

Further, by updating the displacement vector prediction function with the accumulated information, more accurate prediction can be made, which greatly contributes to the accuracy and safety of the surgical operation.

### 4. Fourth Embodiment

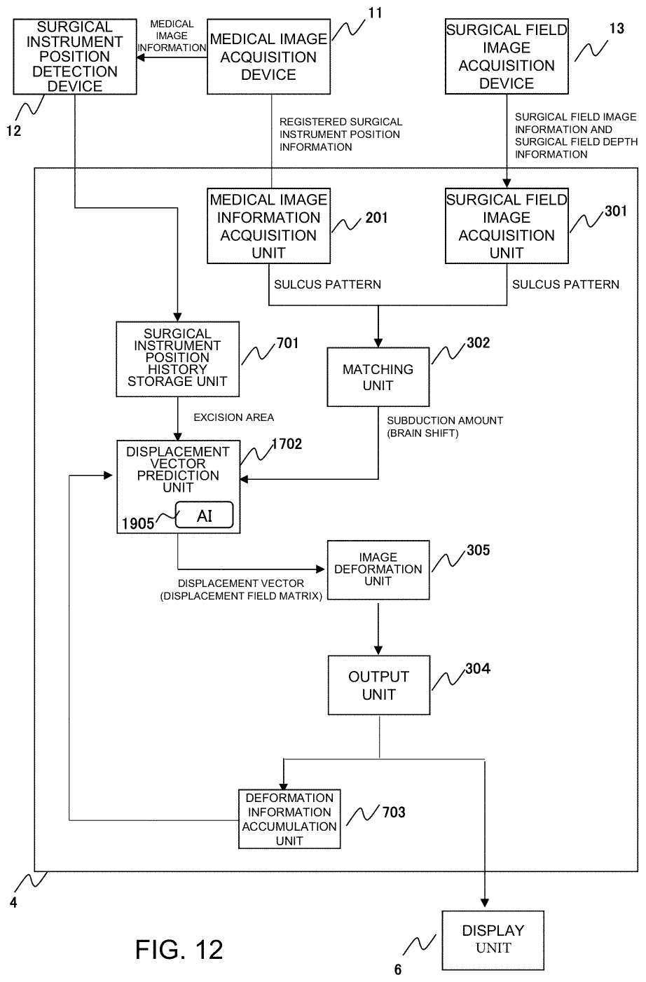

A surgical navigation system of a fourth embodiment will be described with reference to FIGS. 12 to 14.

The surgical navigation system of the fourth embodiment is a configuration for predicting the displacement vector as in the third embodiment, which differs from the third embodiment in that more accurate prediction is performed by using the depth information of the surgical field image (microscopic image) when making the prediction. A displacement vector prediction function is updated after comparing accumulated displacement vector prediction information with microscope image information.

That is, the surgical navigation system of the fourth embodiment receives a captured preoperative image of a brain of a patient before a surgical operation while also receiving position data of a surgical instrument in chronological order, and calculate a range of a living tissue removed by the surgical instrument as an excision area from the chronological position data of the surgical instrument. The surgical navigation system also receives the surgical field image from the surgical field image capturing device that captures the surgical field image of the brain of the patient during the surgical operation, thereby obtaining the depth information relating to a depth up to the brain surface of the surgical field. The surgical navigation system calculates the subduction amount (brain shift) of the brain surface after the surgical operation of the surgical field image from the position coordinates of the brain surface of the preoperative medical image and the depth information relating to the depth up to the brain surface of the surgical field image. The surgical navigation system uses the excision area and the subduction amount to obtain a displacement vector indicating the deformation that occurs in the preoperative image by calculation, and deforms the preoperative image by the obtained displacement vector. The surgical navigation system displays the preoperative image deformed by the image deformation unit on the connected display device.

### 4-1. Configuration

The information acquisition and processing unit 4 of the surgical navigation system includes, as shown in FIG. 12, the surgical instrument position history storage unit 701 that acquires registered surgical instrument position information and medical images from the surgical instrument position detection device 12, a displacement vector prediction unit 1702 that predicts the displacement vector based on the trajectory of the surgical instrument, the surgical field image acquisition unit 301 that acquires the surgical field image from the surgical field image acquisition device 13, the matching unit 302 that compares the sulcus patterns of the medical images and the surgical field images, the calculation unit 303 that calculates the matching result, the image deformation unit 305 that transforms the image, the output unit 304, and the deformation information accumulation unit 703 that accumulates the deformation information.

The displacement vector prediction unit 1702 uses the preoperative image (for example, an MRI image), the removed (excised) area, and the subduction amount (brain shift) as input data, a displacement field matrix as teacher data and is equipped with a learned learning model (artificial intelligence algorithm) 1905. Accordingly, the displacement vector prediction unit 1702 can predict the deformation by inputting an actual preoperative image (medical image 51), an excision area calculated from a trajectory of the surgical instrument position, and the calculated subduction amount (brain shift) 83 into the learning model 1905, and output the displacement field matrix.

### 4-2. Processing

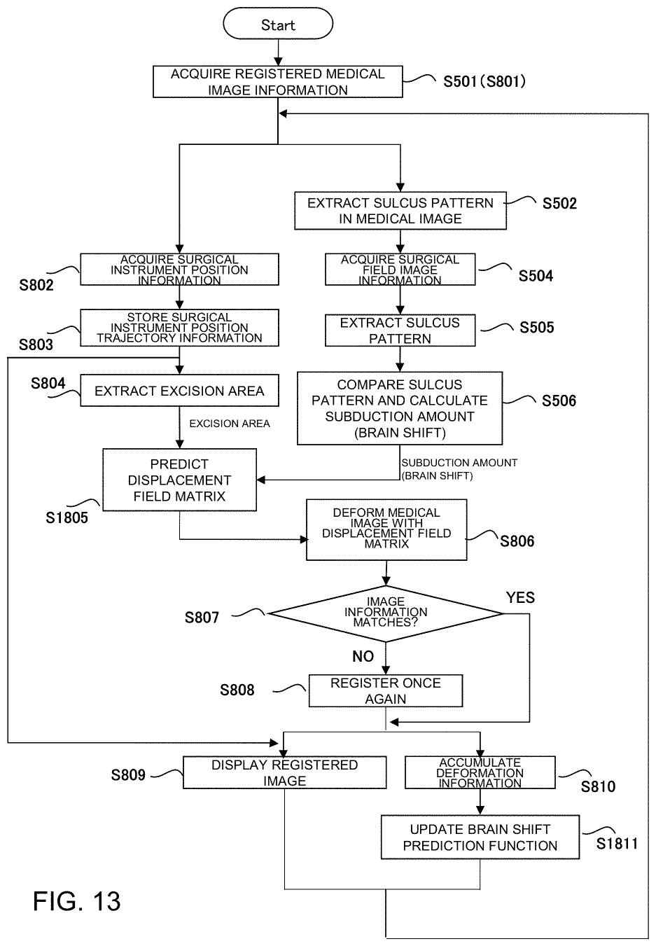

Hereinafter, the processing operation of each unit of the information acquisition and processing unit 4 will be specifically described with reference to a flow of FIG. 13. The same processing as those described in the first to third embodiments is denoted by the same step numbers and will be briefly described.

### (Steps S501 to S502)

The medical image information acquisition unit 201 acquires the medical image 51 in which the medical image space coordinates and the real space coordinates have already been registered with each other from the medical image acquisition device 11, and acquires the sulcus pattern on the medical image 51.

### (Steps S504 to S506)

The surgical field image acquisition unit 301 acquires the surgical field images from the surgical field image acquisition device (surgical microscope) 13, and extracts the sulcus pattern of the surgical field image.

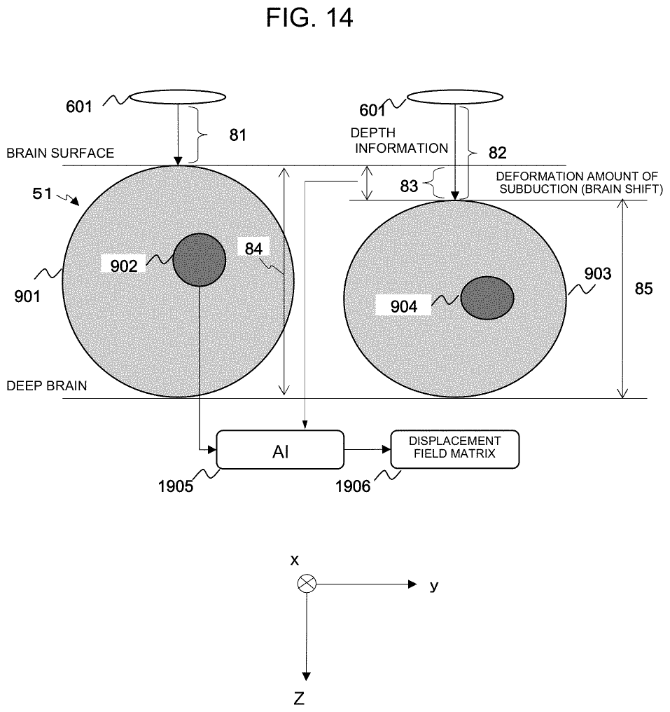

The matching unit 302 compares the sulcus pattern of the medical image 51 with the sulcus pattern of the surgical field image 52, and searches for the range 53 of the medical image 51 that best matches the sulcus pattern of the surgical field image 52. The matching unit 302 obtains the depth information 81 of the preoperative surgical field image 51 and the depth information 82 of the intraoperative surgical field image 52, and calculates the difference (subduction amount: brain shift) 83 between the two images (FIG. 14).

### (Steps S802 to S804)

The surgical instrument position history storage unit 701 acquires the surgical instrument position information from the surgical instrument position detection device 12, stores the surgical instrument position information as a trajectory, and calculates the excision area according to the trajectory information.

### (Step S1805)

The displacement vector prediction unit 1702 inputs the subduction amount (brain shift) 83 calculated in step S506, the excision area 902 calculated in step S804, and the medical image 51 acquired in step S801 into the learned learning model 1905 to obtain a displacement field matrix 1906 to be output by the learned learning model 1905 (see FIG. 14).

### (Steps S806 to S810)

The image deformation unit 304 deforms the medical image 51 by applying the displacement field matrix 1906 obtained in step S1805 to the medical image 51, and obtains the medical image after the brain shift.

When it is determined that the deformed medical image produced in step S806 and the medical image 51 acquired in step S801 do not match, the image deformation unit 305 of the information acquisition and processing unit 4 registers the space coordinates and the real space coordinates of the medical image 51 with each other once again. The output unit 304 superimposes a mark showing the position of the surgical instrument on the registered medical image.

Further, the deformation information accumulation unit 703 accumulates the image deformation information acquired in step S806.

### (Step S811)

The displacement vector prediction unit 1702 obtains a displacement field matrix to match the image captured by the medical image capturing device such as an MRI after the surgical operation with the medical image 51 accumulated in step S810, and relearns the learning model 905 using this displacement field matrix as the output data (teacher data). The input data are the medical image 51, the excision area obtained in step S804, and the subduction amount (brain shift) 83 obtained in step S506.

### 4-3. Effects

According to the fourth embodiment, the following effects can be obtained.

When predicting the displacement vector, it is possible to make a more accurate prediction by comparing the medical image with the visual field image (microscopic image), which can contribute to a highly accurate surgical operation.

What is claimed is:

1. A surgical navigation system, comprising:
a preoperative sulcus pattern extraction unit configured to receive a captured preoperative image of a brain of a patient before a surgical operation and extract a sulcus pattern included in the preoperative image;
a surgical field sulcus pattern extraction unit configured to receive a surgical field image from a surgical field image capturing device configured to capture the surgical field image of the brain of the patient during the surgical operation, and extract a sulcus pattern included in the surgical field image;
a search unit configured to search for a range of a sulcus pattern that matches the sulcus pattern of the surgical field image among sulcus patterns in the preoperative image;
a conversion vector calculation unit configured to calculate a conversion vector that matches the sulcus pattern in the searched range with the sulcus pattern of the surgical field image; and
a calculation unit configured to convert coordinates of the preoperative image by the conversion vector and display the preoperative image on a connected display device.

2. The surgical navigation system according to claim 1, wherein
the sulcus pattern of the surgical field image or the preoperative image is a set of points whose depth information indicating a depth of a brain surface shown in the image is larger than a predetermined value.

3. The surgical navigation system according to claim 2, wherein
the preoperative sulcus pattern extraction unit obtains an average value of the depth information of the preoperative image, and extracts a position where a difference between the depth information and the average value is larger than a predetermined value as a point where a sulcus exists.

4. The surgical navigation system according to claim 2, wherein
the surgical field image capturing device includes a left camera and a right camera,
the surgical field sulcus pattern extraction unit calculates a distance to the brain by using a distance between the left and right cameras, a focal length, and a parallax of images captured by the left and right cameras, respectively, to acquire three-dimensional position information of the surgical field image and obtain an average value of the depth information of the three-dimensional position information, and a position where the difference between the depth information and the average value is larger than the predetermined value is extracted as the point where a sulcus exists.

5. A surgical navigation system, comprising:
a preoperative sulcus pattern extraction unit configured to receive a captured preoperative image of a brain of a patient before a surgical operation and extract a sulcus pattern included in the preoperative image;
a surgical field sulcus pattern extraction unit configured to receive a surgical field image from a surgical field image capturing device configured to capture the surgical field image of the brain of the patient before and during the surgical operation, and extract a sulcus pattern included in the surgical field image;
a search unit configured to search for a range of sulcus patterns that matches the sulcus pattern of the surgical field image among the sulcus patterns in the preoperative image;
a brain shift calculation unit configured to obtain a first depth of the range of the preoperative image from a predetermined position of the surgical field image which is captured before the surgical operation, a second depth of the range of the preoperative image from the predetermined position of the surgical field image which is captured during the surgical operation, and a brain shift which is a difference between the first depth and the second depth;
a displacement vector calculation unit configured to calculate a displacement vector that matches the preoperative image with the surgical field image by deforming the preoperative image in the depth direction with the brain shift;
an image deformation unit configured to deform the preoperative image with the displacement vector; and
a calculation unit configured to display the preoperative image after deformation by the image deformation unit on a connected display device.

6. A surgical navigation system, comprising:
a preoperative image acquisition unit configured to receive a captured preoperative image of a brain of a patient before a surgical operation;
a surgical instrument position acquisition unit configured to receive position data of a surgical instrument in chronological order;
an excision area calculation unit configured to calculate a range of a living tissue removed by the surgical instrument as an excision area from the chronological position data of the surgical instrument;
a displacement vector prediction unit configured to predict a displacement vector indicating deformation that occurs in the preoperative image when the excision area has been excised in the brain by calculation based on the excision area and the preoperative image;
an image deformation unit configured to deform the preoperative image by the obtained displacement vector; and
a calculation unit configured to display the preoperative image after deformation by the image deformation unit on a connected display device.

7. The surgical navigation system according to claim 6, further comprising:
a brain shift calculation unit configured to receive the surgical field image from a surgical field image capturing device that captures the surgical field image of the brain of the patient during the surgical operation to obtain depth information relating to a depth up to a brain surface of the surgical field image, and calculate a subduction amount of the brain surface after the surgical operation of the surgical field image based on the depth information relating to a depth up to the brain surface of the surgical field image and the position coordinates of the brain surface of the preoperative image, and
the displacement vector prediction unit is configured to predict the displacement vector by calculation based on the subduction amount in addition to the excision area and the preoperative image.

8. The surgical navigation system according to claim 6, wherein
the image deformation unit includes a learned learning model in which the excision area and the preoperative image are used as input data, and the displacement vector is used as teacher data.

9. The surgical navigation system according to claim 7, wherein
the image deformation unit includes a learned learning model in which the excision area, the preoperative image and the subduction amount of the brain surface are used as input data, and the displacement vector is used as teacher data.

10. An information processing device, comprising:
a preoperative sulcus pattern extraction unit configured to receive a captured preoperative image of a brain of a patient before a surgical operation and extract a sulcus pattern included in the preoperative image;
a surgical field sulcus pattern extraction unit configured to receive a surgical field image from a surgical field image capturing device configured to capture the surgical field image of the brain of the patient during the surgical operation, and extract a sulcus pattern included in the surgical field image;
a search unit configured to search for a range of a sulcus pattern that matches the sulcus pattern of the surgical field image among sulcus patterns in the preoperative image;
a conversion vector calculation unit configured to calculate a conversion vector that matches the sulcus patterns in the searched range with the sulcus pattern of the surgical field image; and
a calculation unit configured to convert coordinates of the preoperative image by the conversion vector and display the preoperative image on a connected display device.

11. An information processing method, comprising:
receiving a captured preoperative image of a brain of a patient before a surgical operation and extract a sulcus pattern included in the preoperative image;
receiving a surgical field image from a surgical field image capturing device configured to capture the surgical field image of the brain of the patient during the surgical operation, and extract a sulcus pattern included in the surgical field image;
searching for a range of a sulcus pattern that matches the sulcus pattern of the surgical field image among sulcus patterns in the preoperative image;
calculating a conversion vector that matches the sulcus pattern in the searched range with the sulcus pattern of the surgical field image; and
converting the coordinates of the preoperative image by the displacement vector and displaying the preoperative image on a connected display device.
